(19) United States
(12) Patent Application Publication
(10) Pub. No.: US 2020/0205923 A1
(43) Pub. Date: Jul. 2, 2020

(54) MODULAR ROBOTIC SYSTEM USING INTEGRATED CARD MODULES AND/OR HIGH MOBILITY WHEELS

(71) Applicant: TransEnterix Surgical, Inc., Morrisville, NC (US)

(72) Inventors: Kevin Andrew Hufford, Cary, NC (US); Raul Blanco Sanchez, Milan (IT)

(21) Appl. No.: 16/732,308

(22) Filed: Dec. 31, 2019

Related U.S. Application Data
(60) Provisional application No. 62/787,251, filed on Dec. 31, 2018.

(51) Int. Cl. A61B 50/13; A61B 34/30

(52) U.S. Cl. CPC A61B 50/13; A61B 2034/742; A61B 34/30

(57) ABSTRACT

A robotic manipulator arm comprises a base, a robotic manipulator arm on the base, a surgical instrument removably attachable to the robotic manipulator arm. The base is mounted on a plurality of holonomic wheel to allow movement of the manipulator arm in any desired direction.

Some surgical robotic systems use a plurality of robotic arms. Each arm carries a surgical instrument, or the camera used to capture images from within the body for display on a monitor. In some systems, each arm is carried on a separate base. See, for example, the robotic arm 100 of FIG. 3 and those of the Senhance Surgical System marketed by TransEnterix Surgical, Inc. Other surgical robotic systems include multiple robotic manipulator arms on a common base. Still others use a single arm that carries a plurality of instruments and a camera that extend into the body via a single incision. Each of these types of robotic systems uses motors to position and/or orient the camera and instruments and to, where applicable, actuate the instruments. Typical configurations allow two or three instruments and the camera to be supported and manipulated by the system. Input to the system is generated based on input from a surgeon positioned at a surgeon console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

It is often necessary to move the robotic manipulator within the operating room, or between operating rooms. Because of this, the base of the robotic arm requires a high amount of mobility even if it is bulky and or heavy. This application describes an improved robotic manipulator system that may be more easily moved across the floor by users.

The necessary configuration of the manipulator arms can vary greatly depending on the surgical procedure to be performed. This application describes a robotic system that has great flexibility in the positioning of the manipulator arms so as to allow great flexibility for a variety of surgical procedures.

### BRIEF DESCRIPTION OF THE DRAWINGS

FIG. 1 is a perspective view showing a robotic arm on an arm cart. The arm is shown with a surgical instrument mounted on it.

FIG. 2 is a top plan view of a surgical table, and three robotic arm carts of the type shown in FIG. 1 in close proximity with the surgical instruments extending towards the surgical table.

FIG. 3 is a perspective view of one type of robotic manipulator arm;

FIG. 4 is a perspective view of a Mecanum wheel;

FIG. 5 is a top plan view of the manipulator arm of FIG. 3, illustrating available directions of translational motion when the manipulator arm is carried by Mechanum wheels;

FIG. 6 is similar to FIG. 3 but shows available rotational motion;

FIG. 7 is a flow diagram illustrating a method of controlling the Mechanum wheels on a robotic manipulator cart.

### DETAILED DESCRIPTION

As a first embodiment, this application describes a surgical system comprising multiple cart modules 10, each designed to carry one or more robotic arms 12. The cart modules are shaped to allow them to be placed very close together—even so close as to approximate being mounted to the same monolithic base. This arrangement provides the following advantages: (1) the stability of a single, heavy base, (2) the ability of the arms to operate in closely-spaced trocars, and (3) to work together in small and/or deep cavities. The use of a central control module/node on a cart module in such a configuration without an arm mounted above it is also within the scope of the invention.

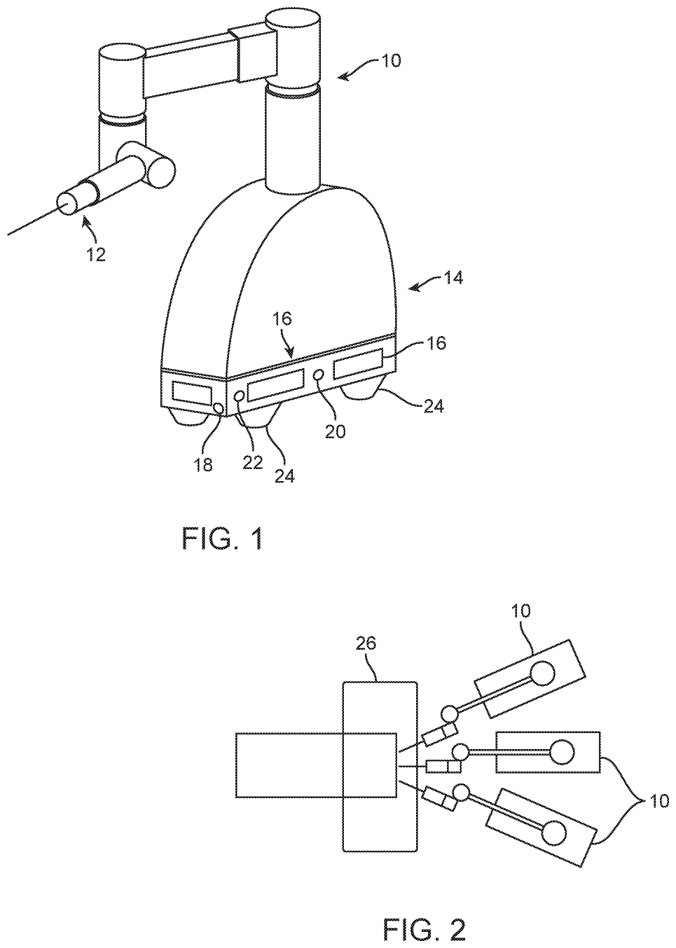

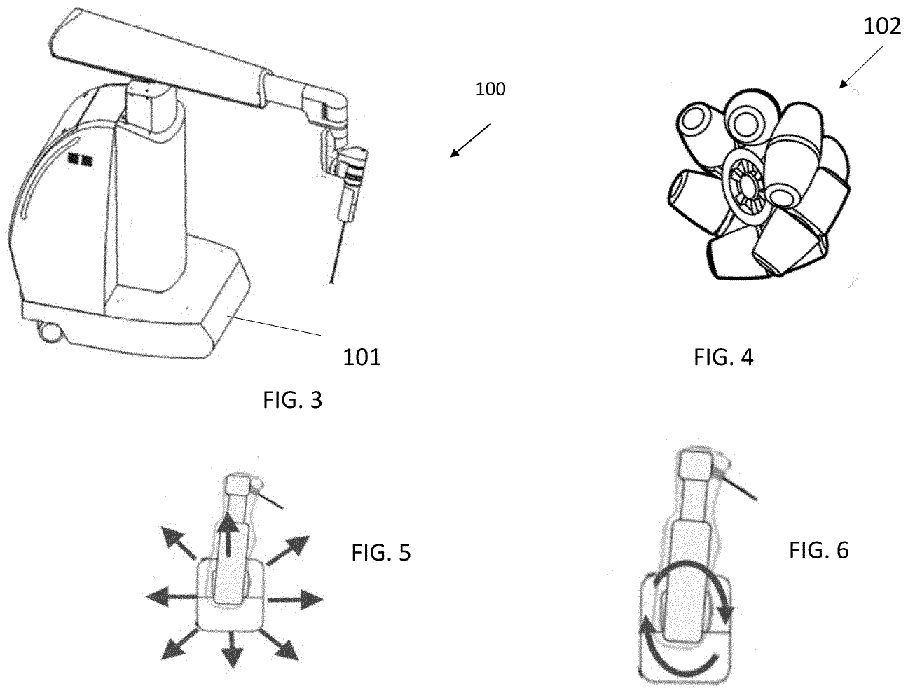

The shape of the carts may be chosen to facilitate positioning of separately mounted arms in close proximity. For example, as best understood with reference to FIG. 1, the lateral cross-section of the base 14 may have the shape of a rectangle, regular trapezoid or wedge, such that the long-edges of adjacent bases may be placed relatively close together. In some embodiments, those edges optionally include planar faces as shown. See FIGS. 1 and 2. Suitable shapes allow individual arms to be closely positioned to one another, as shown in FIG. 2. Note that in this figure the carts have a lateral cross-section that is generally wedge-shaped, allowing the long edges of the wedges to be positioned side-by-side, the narrowest dimension of the wedges closed to the patient bed 26.

The approximation of each of the cart modules may be accomplished in a variety of ways. Mating features or mechanical locking mechanisms 16, which may be manual, electromechanical, electromagnetic, or any combination of the above, may aid in integrating the cart modules together once placed in a chosen arrangement.

Sensors 20 may be used instead, or in addition to, mating features and/or locking mechanisms to provide assurance of a proper connection between adjacent carts or to provide information of the relative position between cart modules. More specifically, sensors along the side of the base can detect the proximity of the base to an adjacent cart module.

The wheels or casters 24 for each cart module/robot base may be holonomic, allowing the module to move in any direction, or some casters may be holonomic while others are not. These casters may be motorized, power-assisted, or manual. This docking or moving may be controlled by an operator. Automatic motion of cart modules—integrated or separate—around the OR, during setup/teardown and/or during the procedure may also be included.

There may be lift mechanisms to lower the cart onto rigid posts or to lower rigid lift points to stabilize the cart and/or remove/reduce the weight on the casters.

Especially for use of an isolated module, stabilizers may extend or emanate beyond the footprint of the base to reduce the need for counterweights and to reduce tipping hazards.

Communications between modules may be accomplished via a variety of means 18, including, but not limited to, electrical contact, optical transmitters and receivers, fiber optic connections. To minimize cabling needs across the floor, this inter-cart communication may be used to amalgamate information from multiple cart modules and just require a single uplink connection (optical fiber, wireless, optical wireless, etc.) to the surgical system's surgeon console or central system node. This communication arrangement may change automatically or manually to accommodate new configurations.

Sensors 18 may be included to detect the presence of obstacles or humans around the robot or cart module. Distance measuring sensors may be used to provide setup assistance—for optimal positioning of the cart from the patient bed 26, for instance. The detection of humans immediately adjacent to the robot may cause an alternate mode of operation and/or with reduced speed or contact force limits for safety.

A second embodiment comprises at least one surgical robotic manipulator, and optionally a plurality of manipulators, mounted on a base. The base may have any of the features described above, or it may be provided without the features of the first embodiment that allow for close spacing of the arms. The base is supported on a plurality of holonomic, or Mecanum, wheels. An example of a Mecanum wheel is shown in FIG. 4. This type of wheel is comprised of a wheel hub 104 and a plurality of rollers 106, each mounted 45 degrees from the axis of the hub. As shown in FIG. 5, the wheels allow the manipulator to be moved across the floor any direction, including side-side, forward-backwards, diagonally etc. They also allow the manipulator to be rotated about the central axis of the base as shown in FIG. 6. This allows hospital personnel to position the robot in the surgery room in a very efficient manner.

The wheels 102 may be controlled through four independent motors on the robot base. The robot controls each wheel motion independently to move the base in the direction desired by the operator. Different methods can be used to acquire operator desired motion as for example (but not limited to) the use of a joystick or a force control sensor on the robot base or arm (see, for example, co-pending application U.S. Ser. No. 16/236,613, Force Based Gesture Control of Robotic Surgical Manipulator, which is incorporated herein by reference). The control algorithm controls the motion of the arm across the floor by controlling:
The desired angle of motion
The desired magnitude of motion
The desired rotation of motion

These parameters can be computed taking into account the wheel kinematics and selecting the correct speed for each wheel.

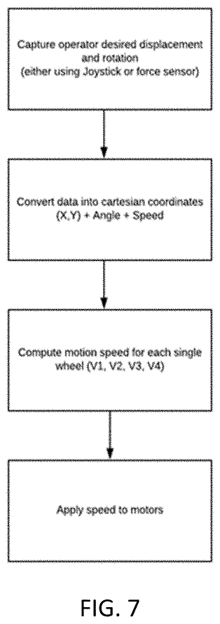

The flow chart of FIG. 7 provides an example on how the motion control can be performed when four wheels are used to perform motion on the robot base, although it should be understood that the invention is not limited to the use of four wheels. Different numbers of wheels may be used.

This type of technology allows the robot base to move freely in all directions helping the position of the medical robot on the surgery room. In an alternative embodiment, the wheels may be included on the surgeon console to allow it to be easily repositioned or moved to a different operating room.

1. A surgical robotic system comprising a plurality of moveable arms carts, each supporting at least one robotic manipulator arm, each arm cart having a base shaped to be positionable proximally adjacent to a base of another one of the arm carts.

2. The system of claim 1, wherein each arm cart has a base having an elongate lateral edge positionable proximally adjacent an elongate lateral edge of another one of the arm carts.

3. The system of claim 1, wherein at least one of the arm carts is releasably attachable to a second one of the arm carts.

4. The system of claim 3, wherein at least one of the arm carts includes a sensor for use in detecting attachment or detachment of the arm carts.

5. The system of claim 1, wherein at least one of the arm carts includes a sensor for use in detecting proximity of arm carts.

6. A robotic manipulator arm comprising:
a base;
a robotic manipulator arm on the base;
a surgical instrument removably attachable to the robotic manipulator arm;
wherein the base is mounted on a plurality of holonomic wheels.

7. The system of claim 1, wherein at least one of the carts has a base mounted on a plurality of holonomic wheels.

8. The system of claim 7, wherein each of the carts has a base mounted on a plurality of holonomic wheels.
